(19) United States
(12) Patent Application Publication
(10) Pub. No.: US 2005/0215909 A1
(43) Pub. Date: Sep. 29, 2005

(54) Electric field control for capacitive micromachined ultrasound transducers

(75) Inventor: Stephen R. Barnes, Bellevue, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/084,451

(22) Filed: Mar. 18, 2005

Related U.S. Application Data
(60) Provisional application No. 60/554,963, filed on Mar. 19, 2004.

(51) Int. Cl. A61B 8/14

(52) U.S. Cl. 600/459

(57) ABSTRACT

Alternating the polarity of the bias voltage in synchrony with the transmit period avoids dielectric and transformer polarization and allows the bias to be changed without generating a pressure artifact as the bias is changed. Alternating the bias polarity may also reduce the bandwidth requirements for square-law operation with low harmonic distortion, allowing more narrow band transmit excitation. Phase-inversion techniques for harmonic or other imaging may be used with a CMUT.

### RELATED APPLICATIONS

The present patent document claims the benefit of the filing date under 35 U.S.C. §119(e) of Provisional U.S. Patent Application Ser. No. 60/554,963, filed Mar. 19, 2004, which is hereby incorporated by reference.

### BACKGROUND

This invention relates to capacitive membrane ultrasound transducers (CMUTs). In particular, the invention relates to the electric field used within electrostatic transducers.

CMUTs generate and receive ultrasound energy. An array of membranes with respective evacuated cavities between the membrane on the surface of a silicon wafer and the silicon substrate are fabricated on silicon wafers using semiconductor processing techniques. Electrodes are deposited on the membrane and the opposing face of the cavity under the membrane. These two electrodes form a capacitor. When the capacitor is charged electrically (or electrically biased), electrostatic forces pull the membrane toward the substrate electrode. In this state, changing the voltage on the capacitor modulates the electrostatic force on the membrane and causes the membrane to move up or down. In a reciprocal fashion, forcing the charged membrane to move up and down changes the voltage on the capacitor.

CMUTs offer many advantages over traditional ceramic transducers. For example, electrostatic transducers may be cheaper to manufacture, allow higher manufacturing yields, provide more size and shape options, use non-toxic materials, and have higher bandwidth. However, electrostatic transducers require a bias voltage for operation. The bias voltage in combination with any transmit voltage is limited to avoid collapse of the membrane. The electrostatic attraction of the membrane cannot exceed the membrane tension. Likewise, the dielectric breakdown of the gap between electrodes is usually avoided. The bias voltage is typically larger than the peak voltage of the transmit voltage to avoid harmonic distortion. This greater bias voltage results in uni-polar excitations. However, a non-zero mean may polarize a magnetic core of a transformer in the transducer or system, possibly distorting operation and resulting in a microphonic response.

CMUTs are square-law devices. Harmonic imaging is difficult with square-law devices. In harmonic imaging, acoustic signals are transmitted at a frequency, and received echoes are isolated for a harmonic of the transmit frequency. It is desired that the received echoes at the harmonic are not a result of a transmitted component at the harmonic frequency. However, a square-law response generates harmonics of the transmitted excitation waveform. Further complicating matters is the bias voltage which sets a non-zero operating point on the square-law response.

The electric fields within a CMUT may be extremely high. For example, a CMUT used for medical ultrasonic imaging may have a cavity height on the order of 0.2 microns and may use bias voltages on the order of 200 Volts. The electric field is thus on the order of 1 GigaVolt per meter. At these electric field intensities, dielectrics are prone to become polarized. Polarized silicon nitride, silicon oxide, gallium arcenide, or other dielectric in the CMUT may act in opposition to the impressed electric field, causing the device to be less sensitive.

### BRIEF SUMMARY

By way of introduction, the preferred embodiments described below include methods and systems for controlling bias and transmit waveforms for a capacitive micromachined ultrasound transducer. Alternating the polarity of the bias voltage in synchrony with the transmit period avoids dielectric polarization and transformer magnetization and allows the bias to be changed without generating a pressure artifact as the bias is changed. Alternating the bias polarity may also reduce the bandwidth requirements for square-law operation, allowing more narrow band transmission. Phase-inversion techniques for harmonic or other imaging may be used.

In a first aspect, a method is provided for controlling bias for a capacitive micromachined ultrasound transducer. First and second sequential acoustic signals are transmitted from the capacitive micromachined ultrasound transducer in a same imaging mode of a same imaging session. A first bias voltage is applied to an element for the transmission of the first acoustic signal, and a second different bias voltage is applied to the element for the transmission of the second acoustic signal. The first and second bias voltages are common along an entire elevation extent of the element.

In a second aspect, a system is provided for controlling bias for a capacitive micromachined ultrasound transducer. The capacitive micromachined ultrasound transducer has a first element. A waveform generator connects with the first element of the capacitive micromachined ultrasound transducer. The waveform generator is operable to generate first and second sequential excitation signals in a same imaging mode of a same imaging session and is operable to apply a different single bias voltage for initiation of the first excitation signal than for initiation of the second excitation signal.

In a third aspect, a method is provided for controlling bias for a capacitive micromachined ultrasound transducer. A bias voltage is applied to the capacitive micromachined ultrasound transducer. An excitation waveform in addition to the bias voltage is applied to the capacitive micromachined ultrasound transducer. The excitation waveform in combination with the bias voltage has positive and negative voltages in a same transmit event. An acoustic waveform is generated as a function of the application of the excitation waveform and the bias voltage. The acoustic waveform has a carrier frequency twice a carrier frequency of the excitation waveform.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for controlling bias voltage for use with a CMUT;

FIG. 2 is a graphical representation showing a transmit voltage and an associated acoustic waveform in one embodiment;

FIG. 3 shows another embodiment of a transmit voltage and an associated acoustic waveform using square waves;

FIG. 4 shows yet another example embodiment of a transmit voltage and an associated acoustic waveform; and

FIG. 5 shows one embodiment of a method for controlling bias voltage on a CMUT.

### DETAILED DESCRIPTION OF THE DRAWINGS AND PRESENTLY PREFERRED EMBODIMENTS

By relying on the square law nature of the device, the transmit voltage is applied with a carrier frequency that is one-half of the desired acoustic pressure waveform. Alternatively, the square root of the desired pressure waveform is applied as the transmit excitation. Square root application results in transmit excitation with a rectified sinusoid. As a result, a sharp negative or positive going peak is provided, introducing distortion from the drive electronics, requiring a greater transmit bandwidth, and limiting available transmitters. Using a transmit excitation that is one-half the frequency of the desired acoustic waveform, the excitation may have a zero mean over multiple cycles. The zero mean more likely avoids waveform asymmetry and associated magnetization of transformer cores or inductors. By providing an excitation waveform with both positive and negative portions, polarization of the dielectric within the CMUT may be limited or avoided.

Alternating bias polarity between positive and negative voltages may avoid dielectric polarization. Since dielectric polarization is a relatively slow process, alternating the polarity in conjunction with the transmit period sufficiently avoids polarization and allows the bias to be changed without generating a pressure artifact as the bias changes. Alteration of the polarity of the bias voltage may also avoid sudden transitions in the generated acoustic waveform due to the square law nature of the CMUT. Accordingly, the acoustic waveform may have less second harmonic or other harmonic energies, allowing for isolation of harmonic information generated through propagation and reflection. Alternating bias polarity may allow use of the CMUT for phase-inversion harmonic imaging. Since the bias may be either a negative or positive polarity, different phases may be provided for different elements. Similarly, a higher bias level is possible. The excitation may progress from a maximum level to a lesser level, avoiding necessity for leaving head room for augmentation of the electric field by the alternating excitation.

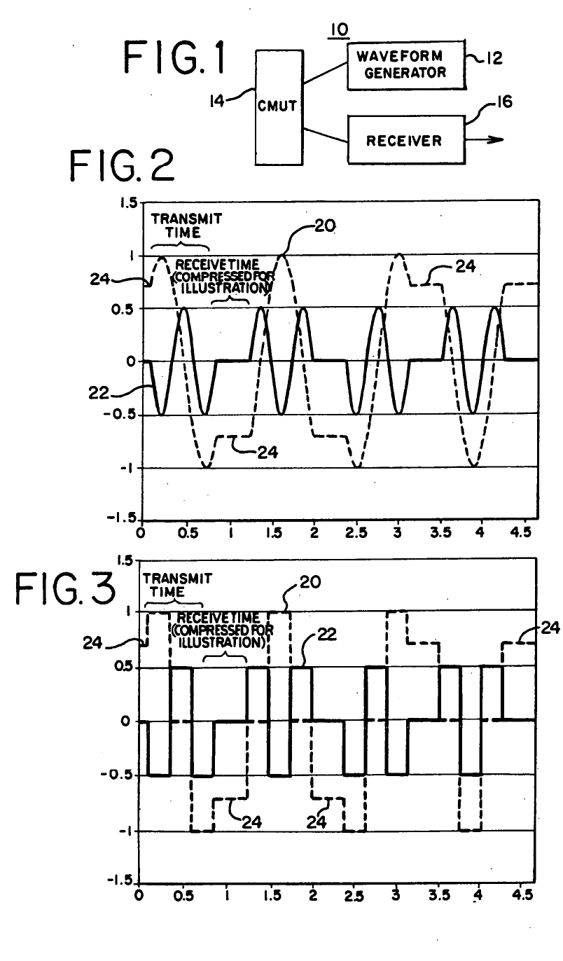

FIG. 1 shows one embodiment of a system 10 for controlling bias for a capacitive micromachined ultrasound transducer (CMUT). The bias is controlled in conjunction with the excitation waveform for minimizing dielectric polarization, magnetization of any transformer core, and/or minimizing transmission of energy at undesired frequencies for harmonic imaging. The system 10 includes a waveform generator 12, a CMUT 14, and a receiver 16. Additional, different or fewer components may be provided. For example, the system 10 is a medical diagnostic ultrasound imaging system and additional components include a detector, scan converter and display for generating ultrasound images. Other ultrasound imaging systems may be provided.

The CMUT 14 is a single or multiple element CMUT array. The elements are arranged in one of various configurations, such as a linear, curved linear, 1.5 dimensional, two dimensional or combinations thereof. CMUTs include any kind of medical ultrasound vibrating acoustic wave transmitters or receivers which use one or more electrostatically charged membranes or structures whose motion is responsive to electrostatic (Coulomb) forces or whose motion results in modulation of electrostatic potential. Such electrostatic transducers include micro-machined, micro-molded or bonded membrane systems used as a transducer. For example, CMUT includes an electrical drivable vibrating micro-diaphragm or membrane made using micro-machining techniques, such as CMOS techniques. On each side of the dielectric gap chamber is a capacitor electrode. In one embodiment, a plurality of doped silicone membranes acts as one electrode and a doped silicone substrate separated from the membranes act as the other electrode. The lateral or largest dimension of the diaphragm or membranes may be in the 50 micron range.

The CMUT 12 is of sufficient bandwidth to pass both fundamental and harmonic components thereof of an acoustical waveform. The size, shape and tension of membrane of other structure may be designed to provide the desired bandwidth. The CMUT 12 may include a mechanical focus, such as an acoustic lens. The CMUT 12 generates an ultrasound transmit beam of acoustic energy or waveforms in response to transmit excitation signals. The acoustic energy propagates outwardly through a subject being imaged. An acoustic beam is formed by propagation of acoustic waves from each of a plurality of elements of the CMUT 12 responsive to respectively delayed and apodized excitation signals. Acoustic energy reflects off of structures. Some of the reflected acoustic energy impinges upon the CMUT 12. In response, the CMUT 12 generates electrical signals for each of the elements.

The waveform generator 12 is a pulser, switches, transistors, memory, digital-to-analog converter, linear transmitter, arbitrary waveform generator, combinations thereof or other now known or later developed device for generating an electrical excitation signal. In one embodiment, the waveform generator 12 is part of a transmit beamformer. A plurality of waveform generators connect through transmit channels to a respective plurality of elements of the CMUT 12. Each channel includes delays, phase rotators and/or amplifiers for relatively delaying and apodizing excitation signals of each channel relative to other channels. The waveform generator 12 is operable to generate an alternating waveform, such as a sinusoidal or square waveform. The excitation signals have peak-to-peak amplitudes of 100, 200 or other greater or lesser voltages.

The waveform generator 12 includes a bias voltage source for supplying a bias voltage to the elements of the CMUT 12. For example, the waveform generator 12 is an arbitrary waveform generator operable to maintain a bias voltage level during receive events. Alternatively, a separate bias voltage circuit is provided for summing with or providing to an element with an alternating excitation signal. The bias voltage circuit allows for two or more selectable bias voltages, such as being an independent waveform generator operable to output a range of different bias voltages. Alternatively, the bias voltage circuit outputs a bias voltage with a few, such as 2, different substantially DC bias voltages. During transmit, the bias voltage establishes an initial position of the membrane or diaphragm of the CMUT 12 pulled partially toward or pushed away from the substrate by electrostatic force. The excitation signal moves the membrane either in or out of the initial position, creating either a rarefaction or compression wave. Higher bias voltages allow for higher possible displacements of the membrane. During receive operation, the bias voltage establishes a charge on the CMUT capacitance so that incoming pressure waves move the membrane in or out, increasing or decreasing the capacitance. The voltage associated with the capacitance is modulated inversely to preserve the relationship Q=CV. The higher the bias, the higher the absolute voltage change on the CMUT 12.

The waveform generator 12 applies an excitation signal and bias voltage to an element of the CMUT 12. The waveform generator 12 is operable to generate sequential excitation signals in a same imaging mode of a same imaging session. Each excitation signal corresponds to a transmit event. For example, a transmit event is followed by a reception event or reception of echo signals in response to the transmission. Each transmit event has a focal region using a transmit aperture. For example, a same elevation aperture is used for different transmit events. Each transmit event is focused at a same focal region at a same elevation angle and/or depth. The transmit events are associated with different azimuth positions or angles to scan a two-dimensional region. A volume is scanned in alternative embodiments. A sequence of transmit events are used to scan a region. A plurality of images are formed from a plurality of scans in a same imaging mode, such as B-mode, color mode, M-mode, spectral Doppler or a combination of modes. An imaging session corresponds to an examination of a patient, such as 5, 10, 15, 30 minutes or other time period examination, for medical diagnosis. Different imaging modes may be used throughout a same imaging session. Each of the excitation signals has a carrier or dominant frequency, such as 1 to 10 megahertz. The excitation signal is a single cycle, plurality of cycles or a fractional number of cycles. For example, 1.5 cycle pulses are generated.

The waveform generator 12 is operable to apply different bias voltages for initiation of the sequential excitation signals. A single bias voltage is applied to each element of the CMUT 12. The bias voltage may be different or the same for different elements. The bias voltage is common along an entire elevation extent of each element. In alternative embodiments, a plurality of different bias voltages are applied substantially simultaneously to different sub-elements of a single element, such as dividing an elevation extent of a element into a plurality of sub-elements for elevation steering or focus.

The different bias voltages used for the sequential excitations or transmit events are different in amplitude, polarity, or both amplitude and polarity. For example and as shown in FIGS. 2, 3 and 4, a excitation signal 20 has an initial positive bias voltage 24 for one transmit event and an initial negative bias voltage for another transmit event. Two or more sequential transmit events may be associated with a same initial and ending bias voltage. The bias voltage 24 is synchronized with the excitation signal 20 to provide a beginning bias voltage that is different than an ending bias voltage during a transmit event. For example, a same amplitude positive bias voltage at the beginning of a transmit event transitions to a same amplitude but opposite polarity negative bias voltage at the end of the transmit event.

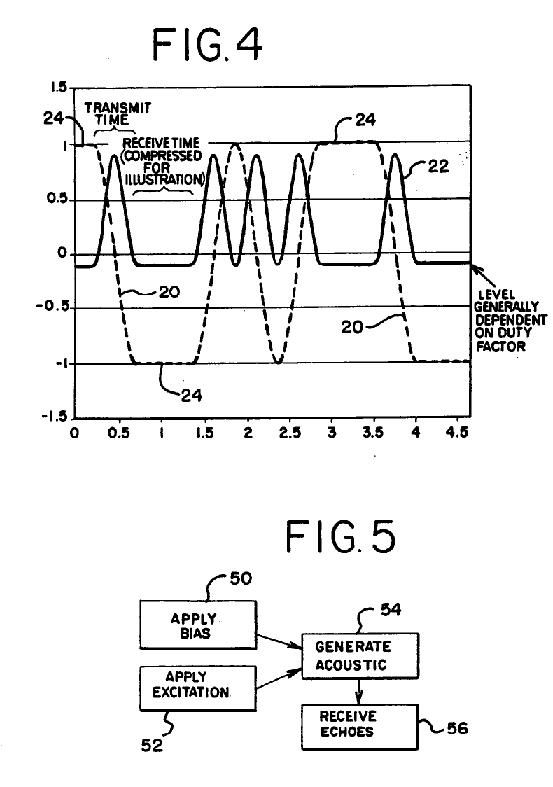

The excitation signal 20 shown in FIG. 3 includes one, minus one and zero voltage levels in addition to the positive and negative bias voltage positions. In any of FIGS. 2, 3 or 4, the receive event bias voltage is the same as the beginning or ending bias voltage for the transmit event. The bias voltage is set to optimize operation for the receive event in one embodiment. For example, FIG. 4 shows the bias voltage at a maximum level for maximum receive event sensitivity. Alternatively, the bias voltage is set based on both considerations of receive sensitivity and the transmit excitation signal as shown in FIGS. 2 and 3.

FIG. 4 shows one embodiment where the bias voltage 24 is set at a maximum positive or negative value. For example, the maximum corresponds to a maximum amplitude allowed by the CMUT 12 while avoiding undesired breakdown voltages. The maximum may be a greatest amplitude used even where the maximum is not near the breakdown voltage. The acoustic pressure waveform 22 is generated in response to transitioning between bias voltages of opposite plurality. Variation with sub-peaks less then the maximum or bias voltage may be provided in alternative embodiments. As shown in FIG. 4, the transmit voltage value of one is a unity number, and may be associated with any of various transmit voltages, such as 100 or 200 volts.

As shown in FIGS. 2 through 4, the bias voltage 20 in combination with the transmit excitation 20 provides both positive and negative voltages in a same transmit event. To take advantage of the square law of nature of the CMUT 12, the alternating current or excitation signal 20 has a similar amplitude as the bias voltage 24. Rather than varying the excitation signal about the bias voltage all in a positive or negative voltage range, the combination of bias voltage 24 and excitation signal 20 includes both negative and positive portions. Accordingly, the square of the excitation signal 20 becomes more dominate such as being a majority of the energy in the resulting acoustic waveform. As shown in FIGS. 2 through 4, the resulting acoustic waveform 22 has twice the frequency of the excitation signal 20 and generally varies around a zero mean. The acoustic waveform 22 may shift up or down depending on the duty factor, such as shown in FIG. 4. Greater or lesser receive time periods relative to the transmit time periods may be provided. The CMUT 14 generates the acoustic signal or waveform 22 in response to the excitation signal 20 and associated bias voltage 24.

The receiver 16 is a receive beamformer, filter, buffer, processor, circuit or other now known or later developed device for forming signals representing different spatial locations from the electrical signals received from the CMUT 12. The receiver 16 connects with the CMUT 12, such as through a transmit and receive switch. As a receive beamformer, the receiver 16 includes analog or digital channels for applying apodization and relative delays or phasing. The relatively delayed and apodized signals from different channels corresponding to different elements of the CMUT 14 are summed to form a sample representing a given spatial location. By dynamically varying the delays, phasing and/or apodization, samples representing one or more scan lines are generated in a receive event responsive to a given transmit event. The summed signals are demodulated to base band. Alternatively, demodulation is performed prior to summation. The demodulation frequency is selected in response to the desired frequency of interest, such as a fundamental or harmonic frequency. Signals associated with frequencies other than mere base band are removed by low pass filtering. As an alternative or in addition to demodulation, band pass filtering isolates the desired information. Using filtering, summation, subtraction or other technique, the receiver 16 is operable to isolate information at a desired frequency band. For example, information at the fundamental transmitted frequency band is isolated. As another example, information for a plurality of frequency bands including or excluding the fundamental is isolated, such as isolating odd harmonics or even harmonics. In yet another example, the receiver 16 is operable to isolate information at a second harmonic of the fundamental transmitted frequency band.

For harmonic imaging, the subject being imaged may include an added contrast agent. The contrast agents may absorb ultrasonic energy at the transmitted fundamental frequency and radiate ultrasonic energy at a second harmonic or other harmonic frequency. As used herein, harmonic includes sub-harmonics, integer harmonics, and fractional harmonics. Generally, harmonic frequencies are frequencies corresponding to non-linear propagation or scattering. As an alternative to contrast agent harmonic imaging, tissue or other structure may be imaged using harmonic frequencies without contrast agent being added during the imaging session.

In addition or as an alternative to filtering to isolate desired information, phase-inversion or other additive or subtractive techniques may be used. For example, data associated with different transmit phases and/or amplitudes is summed or subtracted to obtain information in a desired frequency band. Where the summed signals are 180° out of phase, even harmonic information is isolated from the transmitted pressure signal by adding received signals. Alternating phase is provided by alternating the initial bias polarity, such as shown in FIG. 2. In this example, the number of cycles of the acoustic waveform 22 is an integer number of half cycles of the carrier frequency of the transmitted acoustic waveform 22, but is constrained to exclude any integer divisible by four.

Where phase-inversion second harmonic imaging is not used, a spectrally pure acoustic signal may be formed using the maximum possible bias while still alternating the bias polarity every transmit event. An example is given in FIG. 4. In this example, the number of acoustic cycles is constrained to the series of 1+2n where n is an integer greater than or equal to zero. The maximum possible bias requires the pressure waveform to always start with a rarefaction phase (the electrostatic force is initially reduced from maximum). In order to invert the electric field, the excitation transitions through a half cycle (plus any number of full cycles) to get to the other bias polarity.

FIG. 5 shows one embodiment of a method for controlling bias of a CMUT. The method is implemented using the system 10 shown in FIG. 1 or a different system. The signals and waveform shown in FIGS. 2 through 4 or different excitation signals or acoustic waveforms are used. Additional, different or fewer acts may be provided than shown in FIG. 5 in the same or different order.

In act 50, a bias voltage is applied to a CMUT. Different bias voltages are applied for the transmission of different acoustic signals. The bias voltage at the end of a transmit event has a different amplitude and/or polarity. For example, the bias voltages at the end of two sequential transmit events have substantially the same amplitude but opposite polarity. Bias voltages after the end of the first transmit event is the same or different than the bias voltage applied at the beginning of the sequential transmit event. The bias voltage stays the same or varies during the receive event. As shown in FIG. 4, the bias voltages may range from a maximum amplitude allowed by a CMUT to other amplitude levels between the maximum. In some cases, the reversal of bias polarity may happen every other transmit event, such as shown in FIG. 2. The acoustic waveform 22 of one-half cycle is made with phase inversion. In this case the excitation is one-quarter cycle for each transmit event, and two transmit events are used to transition to the opposite polarity of the excitation waveform.

In Act 52, an excitation waveform is applied in addition to the different bias voltages. “In addition to” includes forming an electrical signal applied to a transducer where the bias voltage is removed during application of an AC waveform. The bias voltage provides a beginning and ending level of the excitation waveform. The bias voltage in addition to the excitation waveform provides an overall waveform used for a transmit event. The excitation waveform and bias voltage provide a square wave as shown in FIG. 3, a sinusoid as shown in FIGS. 2 and 4 or other waveform. During the transmit event, the bias voltage in combination with the excitation waveform provide for a signal with a positive and negative voltages in a transmit event. Alternatively, the resulting acoustic waveform has a carrier frequency twice the carrier frequency of the excitation waveform. In the embodiment shown in FIG. 4, varying the voltage from the maximum positive bias to the maximum negative bias or vice versa generates a half cycle pulse in the acoustic waveform.

In act 54, an acoustic waveform is generated as a function of application of the excitation waveform and bias voltage to the CMUT. The acoustic waveform has a carrier frequency that is about the same or twice the carrier frequency of the excitation waveform. Due to the square-law operation, the acoustic waveform has a substantially uniform polarity despite the excitation waveform having both positive and negative portions. In alternative embodiments, the acoustic waveform has both positive and negative portions in response to the positive and negative portions of the excitation waveforms as shown in FIGS. 2 and 3.

The same or different excitation waveform and associated bias voltage are applied in sequential transmit events. Responsive acoustic waveforms are sequentially generated. By generating acoustic waveforms from a plurality of elements in a same transmit event, an acoustic beam is formed.

In act 56, echo signals are received with the CMUT. The echo signals are received in response to each transmit event. The CMUT transduces acoustic energies of the received echoes into electrical signals. The electrical signals are processed for imaging. Information at a desired frequency band or bands may be isolated for imaging. For example, an excitation signal has a first carrier frequency, such as 1 megahertz. The resulting acoustic waveform generated in the transmit event has twice the carrier frequency, such as 2 Megahertz. For second harmonic imaging, information is isolated at 4 Megahertz. Fundamental, even harmonic, odd harmonic, third harmonic or sub-harmonic imaging is alternatively provided.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for controlling bias for a capacitive micromachined ultrasound transducer, the method comprising:
transmitting first and second acoustic signals at different times from the capacitive micromachined ultrasound transducer in a same imaging mode of a same imaging session; and
applying a first bias voltage to an element for the transmission of the first acoustic signal and a second different bias voltage to the element for the transmission of the second acoustic signal, the first and second bias voltages common along an entire elevation extent of the element.

2. The method of claim 1 wherein transmitting the first and second acoustic signals comprises transmitting the first and second acoustic signals in sequential transmit events.

3. The method of claim 1 wherein applying different bias voltages comprises applying first and second bias voltages with a substantially same amplitude and different polarity.

4. The method of claim 3 wherein applying different bias voltages comprises applying the first and second bias voltages with a substantially maximum amplitude allowed by the capacitive micromachined ultrasound transducer.

5. The method of claim 1 further comprising:
applying an excitation waveform in addition to the different bias voltages to the capacitive micromachined ultrasound transducer, the excitation waveform in combination with the different bias voltages having positive and negative voltages in a first transmit event corresponding to the first acoustic signal.

6. The method of claim 1 further comprising:
applying an excitation waveform in addition to the different bias voltages to the capacitive micromachined ultrasound transducer, the excitation waveform having a first carrier frequency and corresponding to the first acoustic signal;
wherein the first acoustic signal has a second carrier frequency that is about twice the first carrier frequency.

7. The method of claim 6 further comprising:
receiving echo signals with the capacitive micromachined ultrasound transducer; and
isolating information at a harmonic frequency of the second carrier frequency.

8. The method of claim 1 further comprising:
receiving echo signals with the capacitive micromachined ultrasound transducer; and
isolating information at first and second harmonic frequencies of the first and second acoustic signals, respectively.

9. The method of claim 1 further comprising:
applying an excitation waveform in addition to the different bias voltages to the capacitive micromachined ultrasound transducer, the excitation waveform in combination with the different bias voltages being a square wave.

10. A system for controlling bias for a capacitive micromachined ultrasound transducer, the system comprising:
the capacitive micromachined ultrasound transducer having a first element; and
a waveform generator connected with the first element of the capacitive micromachined ultrasound transducer, the waveform generator operable to generate first and second excitation signals at different times in a same imaging mode of a same imaging session and operable to apply a different single bias voltage for initiation of the first excitation signal than for initiation of the second excitation signal.

11. The system of claim 10 wherein the waveform generator comprises an arbitrary waveform generator.

12. The system of claim 10 wherein the capacitive micromachined ultrasound transducer comprises a plurality of elements and wherein the waveform generator comprises a plurality of waveform generators connected with the plurality of elements, respectively.

13. The system of claim 10 wherein the first excitation signal corresponds to a first transmit event and the second excitation signal corresponds to a second transmit event and wherein the different bias voltages comprise first and second bias voltages with a substantially same amplitude and different polarity.

14. The system of claim 13 wherein the substantially same amplitude is at a substantially maximum amplitude allowed by the capacitive micromachined ultrasound transducer.

15. The system of claim 10 wherein the first excitation signal in addition to the different bias voltages have positive and negative voltages in a first transmit event.

16. The system of claim 10 wherein the first excitation signal has a first carrier frequency and wherein a first acoustic signal generated by the capacitive micromachined ultrasound transducer in response to the first excitation signal has a second carrier frequency about twice the first carrier frequency.

17. The system of claim 10 further comprising:
a receiver connected with the capacitive micromachined ultrasound transducer, the element operable to generate an acoustic waveform in response to the first excitation signal, the receiver operable to isolate information at a harmonic frequency of the acoustic waveform from echoes responsive to the acoustic waveform.

18. A method for controlling bias for a capacitive micromachined ultrasound transducer, the method comprising:
applying a bias voltage to the capacitive micromachined ultrasound transducer;
applying an excitation waveform in addition to the bias voltage to the capacitive micromachined ultrasound transducer, the excitation waveform in combination with the bias voltage having positive and negative voltages in a same transmit event;
generating an acoustic waveform as a function of the application of the excitation waveform and the bias voltage, the acoustic waveform having a carrier frequency twice a carrier frequency of the excitation waveform.

19. The method of claim 18 wherein applying the bias voltage and applying the excitation waveform comprises applying a voltage that varies from a maximum positive to a maximum negative or vice versa for each pulse of the acoustic waveform.

20. A method for controlling bias for a capacitive micromachined ultrasound transducer, the method comprising:
transmitting first and second acoustic signals at different times from the capacitive micromachined ultrasound transducer in a same imaging mode of a same imaging session, the first and second acoustic signals for scanning a same elevation aperture; and
applying a first bias voltage to an element for the transmission of the first acoustic signal and a second different bias voltage to the element for the transmission of the second acoustic signal.

21. The method of claim 20 wherein transmitting for a same elevation aperture comprises transmitting focused at a same elevation angle and same depth.

22. The method of claim 20 wherein transmitting for a same elevation aperture comprises transmitting with a same focal region.
